(19) United States
(12) Patent Application Publication
(10) Pub. No.: US 2014/0257105 A1
(43) Pub. Date: Sep. 11, 2014

(54) FLUID FILLABLE CATHETER CAPSULE

(71) Applicant: RESEARCH TRIANGLE INSTITUTE, RESEARCH TRIANGLE PARK, NC (US)

(72) Inventors: David E. Dausch, Raleigh, NC (US); Theodore J. Mosler, Raleigh, NC (US); Edward P. Browka, Chapel Hill, NC (US); David L. Foshee, Apex, NC (US); Jessie Delgado, Durham, NC (US)

(73) Assignee: RESEARCH TRIANGLE INSTITUTE, RESEARCH TRIANGLE PARK, NC (US)

(21) Appl. No.: 14/198,762

(22) Filed: Mar. 6, 2014

Related U.S. Application Data
(60) Provisional application No. 61/773,874, filed on Mar. 7, 2013.

(51) Int. Cl. A61B 8/00; A61B 8/12

(52) U.S. Cl. CPC A61B 8/4281; A61B 8/12
USPC 600/445; 600/458; 600/447

(57) ABSTRACT

A fluid fillable catheter capsule assembly is provided suitable for use with an appropriate catheter in imaging applications for medical or surgical procedures. Methods of constructing such fluid fillable catheter capsule assemblies, methods of using the fluid fillable catheter capsule assemblies, and imaging systems provided with fluid fillable catheter capsule assemblies are provided.

This application claims the benefit of U.S. Provisional Application 61/773,874, filed Mar. 7, 2013 and entitled “Fluid Fillable Catheter Capsule.” The disclosure of U.S. Provisional Application 61/773,874 is hereby incorporated by reference in its entirety, for all purposes.

### FIELD

The present disclosure relates to fluid fillable catheter capsule assemblies suitable for use with an appropriate catheter in imaging systems. The disclosure further relates to methods of constructing such fluid fillable catheter capsule assemblies, methods of filling and using the fluid fillable catheter capsule assemblies, and imaging systems provided with fluid fillable catheter capsule assemblies. Such imaging systems are useful, for example, in ultrasound imaging, particularly in medical or surgical applications.

### DESCRIPTION OF THE RELATED ART

Methods and apparatus for imaging structures within the body of a patient are used widely in the medical field. Such methods and apparatus may utilize a sensor immersed in fluid.

Imaging catheters, for example, typically include a sensor at the distal end of a flexible tubular member which constitutes the catheter body. Ultrasound imaging usually involves use of a catheter body and ultrasound imaging probe assembly. Such assemblies often use an ultrasound probe device that employs one or more transducer elements that are each connected to a pair of electrodes.

Ultrasonic energy produced by the transducer element is readily transmitted by water or saline solution, as well as blood and other body fluids. Ultrasonic energy is not readily transmitted by air. The transducer element in such assemblies may be in a catheter body or portion thereof wherein the catheter body or portion thereof is sealed and fluid filled to allow transmission and reception of acoustic pulses. However, air pockets or bubbles may exist in the fluid which diminishes image quality during use. Any air present in the catheter body in the region of the transducer element must ordinarily be flushed out of the catheter prior to its use since even a very small bubble in the region of the transducer may prevent satisfactory imaging. Flushing the catheter, however, may involve using an undesirable fluid volume without actually ensuring that no bubbles of air remain in the catheter in the region of the transducer element.

In consequence, the art continues to seek improvements in the manufacture, structure and use of imaging catheters.

### SUMMARY

The present disclosure relates to a catheter capsule assembly comprising a capsule having proximal and distal ends and a lumen therethrough, the capsule sealingly attachable at the proximal end to a catheter body and having an opening in the distal end; a flow controller sealingly secured to the inner circumference of the lumen of said capsule proximate to the distal end of the capsule; a sealing element at the proximal end of the capsule, wherein the flow controller and the sealing element are configured to provide a sealable space therebetween, said sealable space arranged to receive a fluid; a passageway in the sealing element in open communication with the sealable space; and a sensor element disposed in the lumen of said capsule in the sealable space.

In another aspect, the disclosure relates to an ultrasound imaging catheter system comprising a catheter capsule assembly having a distal end for insertion into and manipulation within a vascularized organism and a proximal end for attaching to a catheter body and providing a user with control over the manipulation of the distal end of the catheter within the vascularized organism; and a catheter body, wherein the catheter capsule assembly comprises a capsule having proximal and distal ends and a lumen therethrough, the capsule sealingly attached at the proximal end to the catheter body and having an opening in the distal end; a flow controller sealingly secured to the inner circumference of the lumen of said capsule proximate to the distal end of the capsule; a sealing element at the proximal end of the capsule, wherein the flow controller and the sealing element are configured to provide a sealable space therebetween, said sealable space arranged to receive a fluid; a passageway in the sealing element in open communication with the sealable space; and an ultrasound transducer assembly disposed in the lumen of said capsule in the sealable space.

Other aspects, features and embodiments of the invention will be more fully apparent from the ensuing disclosure and appended claims.

### BRIEF DESCRIPTION OF THE DRAWINGS

FIG. 1 is a schematic view of a catheter capsule assembly according to an exemplary embodiment of the present disclosure shown attached to a catheter body.

FIG. 2A is a schematic view of a two part bulkhead according to an exemplary embodiment of the present disclosure.

FIG. 2B is a schematic view of a one part bulkhead according to an exemplary embodiment of the present disclosure.

FIG. 3A is a cross-sectional view of a catheter capsule assembly according to an exemplary embodiment of the present disclosure illustrating the possible positions for a fill tube.

FIG. 3B is a side view of a catheter capsule assembly according to an exemplary embodiment of the present disclosure illustrating the possible positions for a fill tube.

FIG. 3C illustrates a fluid fill assembly according to an exemplary embodiment of the present disclosure.

FIGS. 4A-4L schematically illustrate a method of fabricating a catheter capsule assembly according to an exemplary embodiment of the present disclosure.

### DETAILED DESCRIPTION

In the drawings, it should be understood that when an element is referred to as being proximate another element, it can be directly proximate to the other element or intervening elements may also be present. In contrast, if an element is referred to as being directly proximate to another element, then no other intervening elements are present. Similarly, if an element is referred to as being “connected to” or “coupled to” another element, it can be directly connected to or coupled to the other element or intervening elements may also be present. In contrast, when an element is referred to as being directly connected to or directly coupled to another element, then no other intervening elements are present. As used herein, the term “and/or” includes any and all combinations of one or more of the associated listed items.

As used herein and in the appended claims, the singular forms “a”, “and”, and “the” include plural referents unless the context clearly dictates otherwise. Like numbers refer to like elements throughout.

The disclosure, as variously set out herein in respect of features, aspects and embodiments thereof, may in particular implementations be constituted as comprising, consisting, or consisting essentially of, some or all of such features, aspects and embodiments, as well as elements and components thereof being aggregated to constitute various further implementations of the disclosure. The disclosure correspondingly contemplates such features, aspects and embodiments, or a selected one or ones thereof, in various permutations and combinations, as being within the scope of the present disclosure.

The present disclosure relates to fluid fillable catheter capsule assemblies suitable for use with an appropriate catheter, for example, in ultrasound imaging. It is desirable in ultrasound imaging to provide a fluid path between an ultrasound transducer and the outer wall of the device enclosing the transducer so that acoustic energy is not reflected by air present in the device. It is additionally desirable to minimize fluid volume to prevent bubbles from forming or presenting above the transducer face during use causing degradation in image quality.

As used herein, “catheter” is broadly used to include devices adapted for imaging as well as for applying therapy. Such catheters may include conventional catheters, endoscopes, laparoscopes, transducer devices, probes or other devices adapted for imaging. The catheter capsule assembly of the disclosure may be used with catheters used in, for example, ultrasound imaging catheter technology. In embodiments, the catheter capsule assembly of the disclosure may be used in intracardiac echo (ICE) imaging catheter systems. Such applications may include use of the catheter capsule assembly for image guidance for cardiac ablation, heart valve therapy, tachycardia therapy, or other diagnostic electrophysiology use. In other embodiments, the catheter capsule assembly of the disclosure may be used in intravascular ultrasound (IVUS) imaging catheter systems. Such IVUS systems may be used, for example, in image guidance for stent placement, chronic total occlusion or other vascular diagnostic use.

The present disclosure provides a catheter capsule assembly which minimizes issues with air or bubbles interfering with the image quality by use of a flow controller sealingly disposed in a catheter capsule which provides an enclosed volume for fluid used in imaging or treatment, for example, ultrasound imaging or treatments. In embodiments, the catheter capsule is attachable at a proximal end to a catheter body and has an opening in the distal end. The flow controller is sealingly disposed in the catheter capsule in the distal end proximate to the opening in the distal end of the catheter capsule. In embodiments, the flow controller is a check valve.

In one aspect, the disclosure is directed to a catheter capsule assembly comprising a capsule having proximal and distal ends and a lumen therethrough, the capsule sealingly attachable at the proximal end to a catheter body and having an opening in the distal end; a flow controller sealingly secured to the inner circumference of the lumen of said capsule proximate to the distal end of the capsule; a sealing element at the proximal end of the capsule, wherein the flow controller and the sealing element are configured to provide a sealable space therebetween, said sealable space arranged to receive a fluid; a passageway in the sealing element in open communication with the sealable space; and a sensor element disposed in the lumen of said capsule in the sealable space.

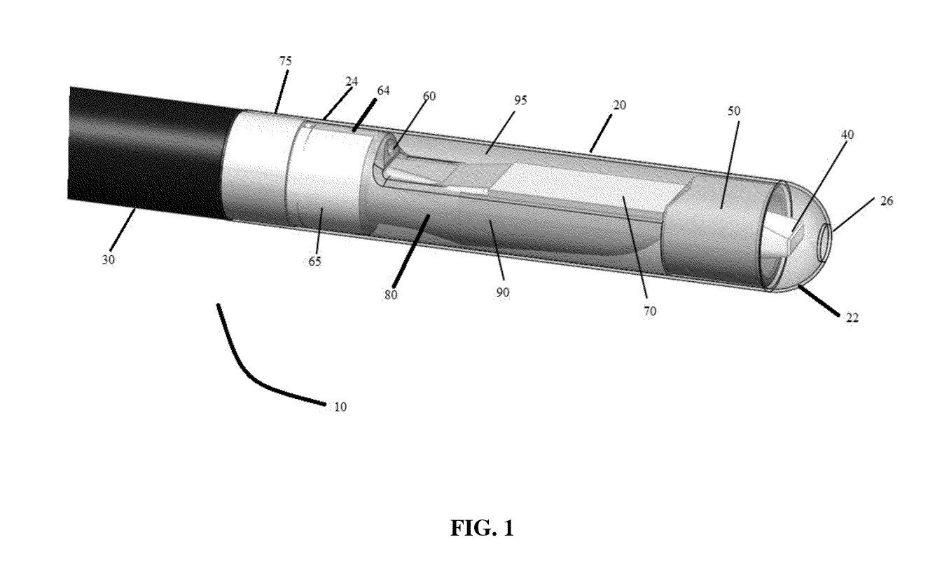

FIG. 1 illustrates a schematic view of a catheter capsule assembly according to an exemplary embodiment of the present disclosure shown attached to a catheter body. As shown, the catheter capsule assembly 10 includes capsule 20 which has a distal end 22 and a proximal end 24. The distal end of the capsule 20 has an opening 26. The capsule 20 is attachable at the proximal end 24 to a catheter body 30. In certain embodiments, the capsule 20 may be sealingly attached to the catheter body 30 through a connector 75.

Capsule 20 has an enclosed volume for retaining fluid in a sealable space 95. Within the enclosed volume is a flow controller which is a check valve 40 positioned in a capsule bulkhead 50 which is sealingly disposed in capsule 20 at the distal end. As shown, the bulkhead 50 is sealingly secured to the inner circumference of the lumen of the capsule proximate to the distal end of the capsule. The check valve is positioned to release air or bubbles that occur in the fluid through the opening 26 of the capsule 20. A sensor element which is a transducer 70 with associated electrical connection 90 is positioned proximate to the bulkhead on the opposite side from opening 26. In the embodiment shown, the transducer 70 positioned proximate to the bulkhead and the electrical connection 90 for the transducer 70 are potted for structural support in potting material 80.

The sealable space 95 in the capsule is adapted to receive a suitable acoustic transmission medium selected from a liquid type fluid. Fluids suitable for use in ultrasound imaging catheter systems are known in the art and include acoustically favorable media such as water or saline solution.

A sealing element 65 is provided in this embodiment as a C-clip with opening 64 which provides a space for a passageway with fluid communication between the sealable space for containing fluid and a fluid source outside the capsule (not shown). The passageway in FIG. 1 is fill tube 60. The fill tube allows the acoustic transmission medium to be delivered into the defined space 95 in capsule 20, preferably shortly prior to use. The sealable space 95 is filled with fluid through fill tube 60 and the check valve 40 operates to release air or gas from the capsule through opening 26.

The catheter capsule assembly of the disclosure comprises a capsule having proximal and distal ends and a lumen therethrough. The capsule is sealingly attachable at the proximal end to a catheter body and has an opening in the distal end. In embodiments, the capsule is a cylindrical shape. The opening in the distal end may be a circular or oval opening or of any other useful shape that permits fluid to exit the distal end of the capsule.

In embodiments, the capsule may be made of any material suitable for medical use which may include contact with body fluids and tissue and which allows for acoustic transparency. A capsule with acoustic transparency may be made of any suitable material with desired acoustic transmission properties, for example, a biocompatible material such as a polyolefin, thermoplastic, thermoplastic elastomer, thermoset or engineering thermoplastic or combinations, copolymers or blends thereof.

As an alternative, the capsule may be made of material which is not acoustically transparent and may comprise an acoustically transparent window in a portion of the capsule overlying the sensor element area of the catheter capsule assembly.

The capsule in embodiments is formed from a biocompatible polymeric material which may be semi-rigid or rigid for contributing to the structural support of the catheter capsule assembly. Biocompatible polymeric materials useful for the capsule may be selected from the group consisting of polyether block amide, silicone, polyurethane, acrylic, acrylonitrile butadiene styrene (ABS), or other suitable materials for catheters or endoscopic probe housings.

The catheter capsule assembly of the disclosure is sealingly attachable at the proximal end to a catheter body. Such catheter body typically is an elongated tubular member having a central bore. Materials useful for catheter bodies are known in the art and include thermoplastic elastomers (TPE) such as thermoplastic polyurethanes, polyester elastomers, silicones and polyamide block copolymers. In embodiments, the material used for the catheter body is a polyether block amide. One suitable material is Pebax™. The capsule may be attached to the catheter housing a heat sealing process. The capsule tubing is placed over the catheter shaft tubing, and a clamshell heat seal tool is placed over the joint to reflow the Pebax tubing and create a uniform, fluid tight seal. A connector may be used in the area of the heat seal to provide additional structural support for the joint. The connector may be made of a metal material that is reflective to X-ray radiation such that the connector functions as a marker band which provides visualization under fluoroscopic imaging. In such embodiments, the connector may be made of stainless steel, platinum or tungsten.

The catheter capsule assembly of the disclosure includes a flow controller sealingly secured to the inner circumference of the lumen of the capsule proximate to the distal end of the capsule. The flow controller may be secured directly to the inner lumen of the capsule or may be secured through use of a structurally supporting member. As used herein, the flow controller may be a fluid control valve such as a one-way or non-return valve. In embodiments, the non-return or one-way valve is a check valve which allows fluid to flow through it in only one direction. The use of a flow controller such as a check valve allows for purging of air or air bubbles in the fluid introduced into the catheter capsule assembly from the capsule out through the opening in the distal end of the capsule. In addition, the flow controller prevents body fluids from entering the sealable space arranged to receive a fluid in the catheter capsule assembly during use. Such back flow of body fluids may be caused by momentary generation of suction which may pull blood back into the catheter capsule assembly. The blood that enters the catheter capsule then clots and causes degradation of the image during use. In addition, changes in blood pressure may cause a fluctuating pressure at the distal end of the catheter capsule assembly which can induce backward flow into the catheter capsule assembly. The use of a flow controller at the distal end of the capsule as disclosed prevents the backward flow of blood or other bodily fluids into the sealable space arranged to receive a fluid in catheter capsule assembly, and protects the integrity of the sensor element therein. The flow controller may be disposed in the distal end of the capsule such that no other feature is present between the opening in the distal end of the capsule and the flow controller. In this embodiment, the flow controller is directly proximate to the opening in the distal end of the capsule. In such embodiment, air in the capsule may be expelled as needed during the filling of the sealable space arranged to receive a fluid or during using of the catheter capsule assembly with a catheter body for medical or surgical procedures.

In embodiments, the flow controller is a fluid control valve. In embodiments, the fluid control valve is a check valve which allows fluid to flow through it in only one direction. As used herein, a check valve is a one-way valve that allows free flow when a positive differential pressure is applied across the valve (i.e., the pressure at the inlet is greater than the pressure at the outlet), but inhibits (or “checks”) backflow when a negative differential pressure is present. The check valve works automatically to allow bubbles or air in the sealable space to flow out of the catheter capsule assembly through the check valve and out of the capsule through the opening in the distal end of the capsule. The check valve may operate, for example, in response to pressure from the flow of fluid into the sealable space. In embodiments, the check valve may allow some amount of fluid to flow through the valve and out the opening in the distal end of the capsule, in addition to air or air bubbles. The check valve may be designed for a specific pressure limit desired based on the features of the catheter capsule assembly and the components thereof, the fluid to be used and/or other requirements based on the desired use of the catheter capsule assembly. Types of check valves that may be used include, for example, a diaphragm check valve, a duckbill check valve, umbrella check valve or in-line check valve.

In one embodiment, the check valve is a duckbill check valve. Duckbill valves are typically one-piece, elastomeric components that act as backflow prevention devices or one-way valves. A duckbill check valve generally has elastomeric lips in the shape of a duckbill which prevent backflow and allow forward flow. A duckbill valve useful in embodiments of the catheter capsule assembly of the disclosure is self contained i.e. the critical sealing function is an integral part of the one piece elastomeric component as opposed to valves where a sealing element has to engage with a smooth seat surface to form a seal.

In embodiments, the flow controller is formed from elastomeric sealing elements that allow forward flow and prevent backflow. Materials particularly useful for the flow controller include medical grade elastomer materials. By way of example, the flow controller may be formed from materials selected from the group consisting of silicone, silicone rubber, polyurethane-based elastomeric composite, and polyisoprene.

The flow controller may be directly secured to the inner circumference of the lumen of the capsule by adhesive or other securing methods or may be secured to the inner circumference of the lumen of the capsule through use of a structurally supporting member. Such structurally supporting member will sealingly engage the flow controller and be sealingly positioned within the distal end of the capsule.

In one embodiment, the structurally supporting member may include a bulkhead which is affixed by adhesive, embedded in potting material or otherwise sealingly positioned within the distal end of the capsule. The bulkhead may be a one piece unit or may be formed of two or more parts including a flow-control bulkhead and a separate sealing element. A two-piece bulkhead may be advantageous as allowing for greater tolerance to variance in the dimensions of the sensor contained in the capsule. Moreover, a two-part design can be made with smaller profile, with no additional material or components adjacent to the sensor that could increase the diameter of the capsule. Therefore, a two-part design may provide the smallest possible catheter diameter.

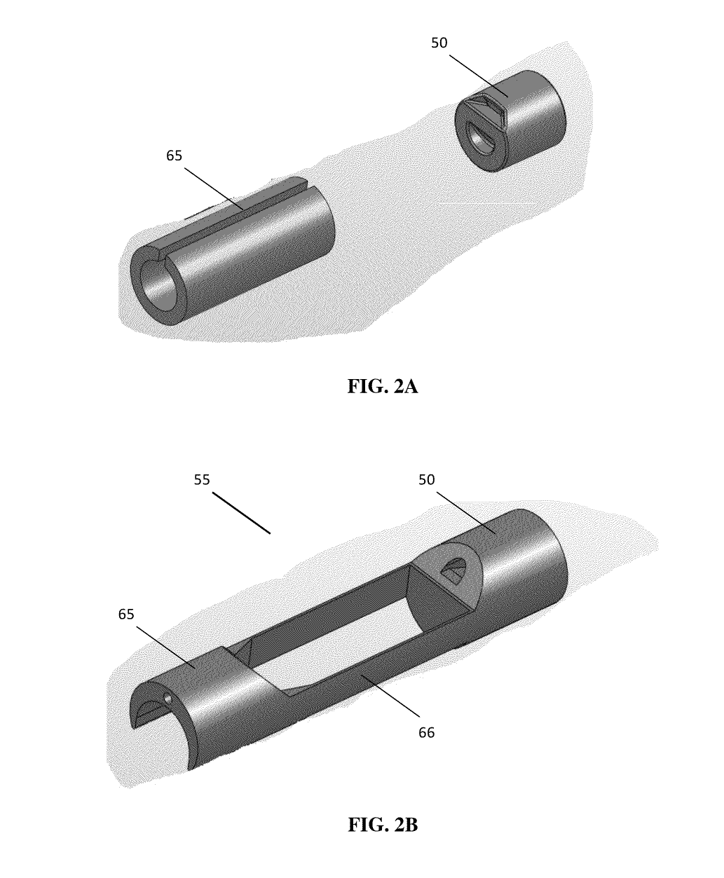

FIG. 2A is a schematic view of a two part bulkhead according to an exemplary embodiment of the present disclosure including a flow-control bulkhead 50 and a sealing element 65. FIG. 2B is a schematic view of a one part bulkhead unit 55 according to an exemplary embodiment of the present disclosure, which combines the flow-control bulkhead 50 and sealing element 65 connected together with bulkhead sidewalls 66. In the embodiment of FIG. 2B, the bulkhead sidewalls 66 may be used for structural support of the flow controller and the sensor element, which may include a sensor, or specifically an ultrasound transducer, and accompanying electrical connectors, or specifically wire bundles, cabling or flex cabling. Either of these embodiments may be sealingly secured to the inner circumference of the lumen of the capsule proximate to the distal end of the capsule to provide a sealed fluid chamber with an integrated flow controller in the capsule. The bulkhead sealingly engaging a flow controller may be used to create a single, simple flow path between the inlet of the passageway in the sealing element and the bulkhead integrated flow controller over the sensor element and a sealable space with minimal volume. This minimal volume reduces the amount of catheter lumen volume that needs to be filled with fluid to fully encapsulate the sensor in fluid without filling the entire catheter volume. Furthermore, any bubbles that are created during the filling process are limited to the minimal fluid volume in the sealable space in the capsule and can be readily cleared away or flushed out by the flow of the fluid around or over the sensor element. Any air bubbles that appear are contained only within the capsule and can be readily flushed out the distal end of the capsule. Moreover, in systems where a larger volume or the entire catheter body is filled with fluid, bubbles may be trapped along the lumen of the catheter body that can migrate after initial filling/flushing and reappear in the capsule during the surgical and/or imaging procedure. These bubbles that reappear may degrade sensor performance, or specifically ultrasound image quality, during the surgical procedure.

Typically the bulkheads of FIG. 2A and FIG. 2B may utilize an adhesive or a potting agent such as an epoxy to create a seal between the sensor element, the bulkhead and the capsule. For a two-part bulkhead, the sensor is placed between the flow-control bulkhead and the sealing element. Fluid fills within the capsule and around the sensor. For a one-part bulkhead, the sensor can additionally be sealed to the bulkhead sidewalls 66 using epoxy such that fluid fills only a smaller volume directly above the sensor face and not in the space under the sensor. The sidewalls are sealed to the capsule tubing to prevent fluid from filling under the sensor.

The bulkhead is positioned in the capsule such that it forms a fluid-tight barrier between the fluid fillable space in the enclosed volume of the capsule and the opening in the distal end of the capsule. The flow controller is supported within the bulkhead and allows for bubbles or air in the fluid fillable space to exit through the opening in the distal end of the capsule. In embodiments, a check valve is sealingly engaged in the bulkhead by use of adhesive, such as a UV curable adhesive. Useful adhesives may include acrylic, cyanoacrylate, silicone epoxy, or other adhesives or epoxies that provide a reliable fluid tight seal to the capsule tubing material.

The check valve in the bulkhead may be disposed in the distal end of the capsule such that no other feature is present between the opening in the distal end of the capsule and the check valve. In this embodiment, the check valve is directly proximate to the opening in the distal end of the capsule. In such embodiment, air in the capsule may be expelled as needed during the filling of the sealable space arranged to receive a fluid or during using of the catheter capsule assembly with a catheter body for medical or surgical procedures.

The catheter capsule assembly of the disclosure includes a sealing element at the proximal end of the capsule with the flow controller and the sealing element configured to provide a sealable space therebetween. The sealable space is arranged to receive a fluid such as an acoustically favorable medium. The sealing element may provide an annular seal at the proximal end of the capsule. In embodiments, a sealing element such as a spring clip, an O ring, a split ring clip or a C clip may be used. In other embodiments, the sealing element may be a machined or molded component in the shape of an O ring or C clip. The machined or molded component may be made of a metal or plastic material. The sealing element includes a passageway therethrough for allowing fluid to flow into the sealable space as needed. Such passageway may be an opening in the connector providing flow communication between the sealable space and a fluid source outside the capsule or a separately formed passageway provided by a tube through the connector which allows flow communication between the sealable space and a fluid source outside the capsule. By way of example, a C clip may be used to provide an opening that operates as a guide feature for the passageway such as a fill tube.

In embodiments, a C clip may be used with adhesive sealing and potting material to maintain a seal at the proximal end of the capsule, providing a sealable space between the proximal end and the flow controller. When a C clip is used, a fill tube may be placed in the opening in the C clip to provide the flow communication therethrough. The fill tube may be attached outside the capsule to a fluid source that allows the sealable space to be filled with fluid as needed.

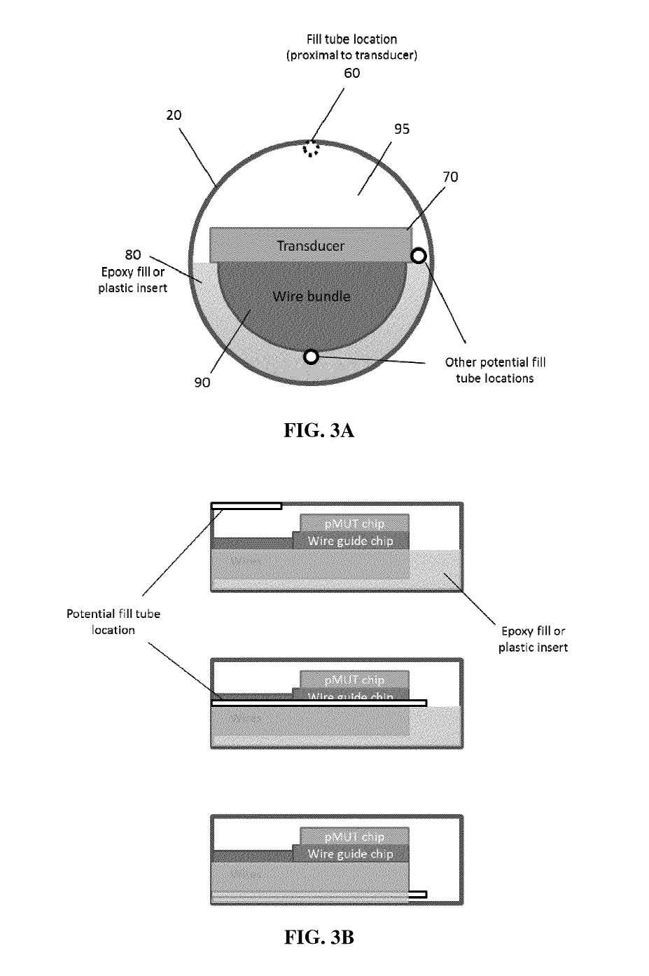

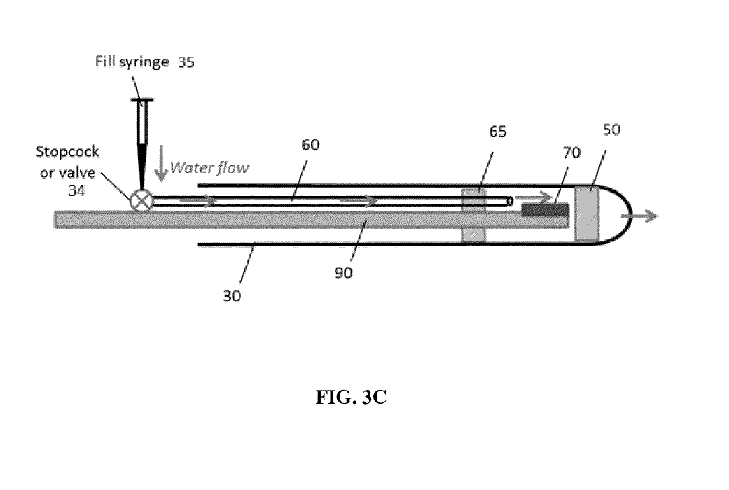

FIG. 3A is a cross-sectional view of a catheter capsule assembly according to an exemplary embodiment of the present disclosure illustrating possible positions for a fill tube. FIG. 3B is a side view of a catheter capsule assembly according to an exemplary embodiment of the present disclosure illustrating possible positions for a fill tube. In a preferred embodiment, the fill tube 60 is proximal to the sensor element 70, such as an ultrasound transducer, such that the space above the transducer is filled with fluid, and the fill tube is not seated directly above the transducer face so as not to impede the acoustic path from the transducer to the body. The ability to minimize the fluid volume needed reduces the possibility that air bubbles will be present in the fluid which may degrade the image obtained by the sensor or transducer element.

A sensor element is disposed in the lumen of said capsule in the sealable space. The sensor element may be any type of unit known for use in medical or surgical procedures, particularly procedures where a sensor element is immersed in fluid. In embodiments, the sensor element will comprise an ultrasound transducer or transducer array and may be part of an ultrasound transducer assembly.

The transducer assembly typically comprises an ultrasound transducer element and an electrical connection including at least one of a wire, coaxial wire, wire bundle, cable or flex cable. The transducer assembly is configured for transmitting acoustic signals, receiving reflected echo signals corresponding to the transmitted acoustic signals, and transforming the received echo signals to electrical signals.

The ultrasound transducer element may include an array of transducers or a single transducer. The one or more transducers may be formed from one or more known materials capable of transforming applied electrical signals to pressure distortions on the surface of the one or more transducers. Suitable materials for such transducers may include piezoelectric ceramic materials including ZnO, AIN, LiNbO₄, lead antimony stannate, lead magnesium tantalate, lead nickel tantalate, titanates, tungstates, zirconates, or niobates of lead, barium, bismuth, or strontium, including lead zirconate titanate (Pb(ZrₓTi₁₋ₓ)O₃ (PZT)), lead lanthanum zirconate titanate (PLZT), lead niobium zirconate titanate (PNZT), BaTiO₃, SrTiO₃, lead magnesium niobate, lead nickel niobate, lead manganese niobate, lead zinc niobate, or lead titanate; piezocomposite materials; or piezoelectric polymer materials such as polyvinylidene fluoride (PVDF), polyvinylidene fluoride-trifluoroethylene (PVDF-TrFE), or polyvinylidene fluoride-tetrafluoroethylene (PVDF-TFE), for example. Other transducer technologies may be used, such as composite materials, single-crystal components, and semiconductor devices such as capacitive micromachined ultrasound transducers (cMUT) or piezoelectric micromachined ultrasound transducers (pMUT).

Exemplary transducers suitable for use in the catheter capsule assemblies of the present disclosure include, for example, those disclosed in U.S. Pat. No. 7,449,821 issued Nov. 11, 2008, U.S. Patent Publication No. 2010/0168583 published Jul. 1, 2010, and U.S. Patent Publication No. 2012/0319535 published Dec. 20, 2012, each incorporated by reference in its entirety for all purposes. In embodiments of the disclosure, pMUT devices are used for intravascular ultrasound (IVUS) and intracardiac echo (ICE) imaging.

The transducer assembly includes an electrical connection 90 to provide connectivity to an external ultrasound system. Electrical connection may be made of individual wires, insulated wires or coaxial wires arranged to form a wire bundle or cable assembly. Alternately, the electrical connection may be made using a flex cable or multiple flex cables comprising one or more flat polymer ribbons with one or more metal traces deposited on the ribbons to carry electrical signals. In use, the transducer assembly will be in electrical communication through a catheter body to the ultrasound system. In embodiments, the electrical connection 90 exits the capsule through the sealing element 65. The space between the electrical connection and sealing element is filled with epoxy or other potting material so as to form a fluid tight seal around the electrical connection within the sealing element.

In an embodiment of the disclosure, the transducer 70 is disposed in the sealable space 95 inside the capsule proximate to the flow control bulkhead 50. In order to provide structural support to the transducer and flow controller within the capsule and minimize unwanted movement of the transducer during use, the transducer and flow controller may be potted prior to positioning in the capsule. FIG. 3A shows an embodiment of a transducer 70 disposed on a wire bundle 90. As shown, the transducer assembly is positioned in the capsule of a catheter capsule assembly by use of adhesive fill such as epoxy fill or by support from a plastic insert.

The potted material may be biocompatible or nonbiocompatible, depending on whether the potted material will come in contact with bodily fluids or tissue. Preferably, the potted material is completely encapsulated within the capsule. The potting material may be epoxy, silicone, polyurethane, acrylic, cyanoacrylate or other suitable material. In one embodiment, the potted transducer and fluid or flow control bulkhead may be attached directly to the capsule tubing using the potting material or using a separate adhesive material to form a fluid tight seal with the capsule tubing. In other embodiments, the transducer may be potted or encapsulated into a one-part bulkhead, and the bulkhead is bonded to the capsule tubing.

Prior to use of the catheter capsule assembly, the assembly is sealingly attached at the proximal end of the capsule to a catheter body. The capsule assembly may contain at least one of a fluid control bulkhead, a flow controller element or valve, a sensor element and electrical connection, a sealing element, a fill tube, a plastic insert, potting epoxy, or adhesive, and the capsule tubing with distal outlet hole. The capsule components are sealingly attached to the capsule tubing such that a sealable space (95) is formed above and/or around the sensor element. For an ultrasound transducer, fluid fill of this sealable space provides a fluid path for acoustic transmission from the transducer face out of the catheter to the body being imaged. Attachment of the capsule assembly to the catheter body may be by any method that provides a fluid tight sealing engagement between the catheter capsule and the catheter body. In one embodiment, a capsule connector (75) may be used to secure the capsule to the catheter body such that the catheter body and/or capsule tubing are attached to the connector using a heat seal or adhesive. The capsule tubing may be heat sealed or reflowed over the catheter housing to form a continuous, fluid tight bond with the connector and catheter housing. The connector may be metal and may also function as a fluoroscopic marker band.

In one embodiment, a catheter capsule assembly according to the present disclosure is used in an ultrasound imaging catheter system useful in medical or surgical procedures. The ultrasound imaging catheter system comprises a catheter capsule assembly having a distal end for insertion into and manipulation within a vascularized organism and a proximal end for attaching to a catheter body and providing a user with control over the manipulation of the distal end of the catheter within the vascularized organism; and a catheter body. The catheter capsule assembly comprises a capsule having proximal and distal ends and a lumen therethrough, the capsule sealingly attached at the proximal end to the catheter body and having an opening in the distal end; a flow controller sealingly secured to the inner circumference of the lumen of said capsule proximate to the distal end of the capsule; a sealing element at the proximal end of the capsule, wherein the flow controller and the sealing element are configured to provide a sealable space therebetween, said sealable space arranged to receive a fluid; a passageway in the sealing element in open communication with the sealable space; and an ultrasound transducer assembly disposed in the lumen of said capsule in the sealable space.

The ultrasound transducer assembly in embodiments may comprise a piezoelectric ultrasound transducer and an electrical connection such as a cable of electrical wires, coaxial wires or flex cable. The electrical connection typically will be of a length suitable to pass through the catheter body when the catheter capsule assembly is attached to the catheter body. Electrical connection length may preferably be from 3 feet to 10 feet long, and may then be connected to an ultrasound control or imaging system or a cable for an ultrasound system. Ultrasound imaging systems and their use are known in the art. More specifically, such a catheter with ultrasound transducer assembly in a fluid filled capsule may particularly apply to intracardiac echocardiography (ICE) catheters with outer diameter of 8F to 15F (2.7 to 5 mm) or intravascular ultrasound (IVUS) catheters with outer diameter of 3F to 8F (1 to 2.7 mm). An ultrasound transducer assembly with fluid-filled capsule may also apply to endoscopic or laparoscopic ultrasound probes for diagnostic gastrointestinal imaging or image guidance of minimally invasive or laparoscopic surgeries. Such endoscopic probes may have diameter of 5 to 15 mm.

When the catheter capsule assembly is attached to the catheter body and is ready for use, an outside fluid source may be connected to the passageway in the sealing element in open communication with the sealable space. The sealable space may then be filled with an acoustically acceptable medium.

FIG. 3C shows an embodiment of the fluid fill assembly, including a syringe 35, valve or stopcock 34 and fill tube 60. The fill tube 60 is placed through the sealing element 65 in the capsule and extends proximally down the length of the catheter body 30. The fill tube may be terminated with a stopcock or other valve containing a luer or other fitting for seating of a syringe. A syringe filled with the appropriate fluid, for example sterile saline, is attached to the stopcock, the stopcock is opened, and fluid is injected using the syringe through the fill tube and into the capsule. Fluid is flushed through the capsule and through the flow controller at the distal end of the catheter until there is no air entrapped in the capsule. If air or bubbles remain after initial fill, more fluid can be injected by pressing and/or pulsing the plunger of the syringe to fully purge the air out of the capsule through the flow controller such as a fluid control valve and exits through the opening at the distal end of the capsule. The process continues until there are no bubbles in the capsule. The capsule tubing may be transparent to allow quick visual inspection of the capsule fluid fill. The fluid control valve self-seals when no syringe pressure is applied such that the fluid remains in the enclosed capsule, and no air can migrate or backflow into the capsule through the one-way fluid control valve. Once the capsule is fluid filled with no air, the stop cock valve is closed to seal the fluid system. This maintains a sealed, fluid filled capsule with no entrapped air. Moreover, if a bubble appears during the procedure and degrades the ultrasound image, the catheter capsule assembly may be quickly re-flushed using the syringe with minimal fluid volume to purge the air bubble, and the fluid control valve self seals to maintain the air-free fluid-fill in the capsule. If sterile saline is used, this may be done during the procedure without removal of the catheter from the patient.

The ability to fill the catheter capsule assembly at the time of use provides a number of advantages such as ability to use an appropriate fluid (e.g. sterile saline) for the procedure and to limit the fluid volume required. Limiting the fluid volume limits the length of the lumen containing fluid, and minimizes volume where air could be entrapped and reemerge into the capsule during the procedure. In addition, the catheter can be quickly and easily filled with fluid, and visual inspection of the catheter tip can be performed instantaneously to determine if additional fluid flushing is required in the capsule to expel entrapped air. In other systems where the entire length of the catheter lumen is filled with air, the entire catheter lumen cannot be inspected, as the catheter body tubing is opaque, and air can be entrapped around the electrical wiring or other structures in the lumen. These systems may require the user to swing the catheter tip around to impart centrifugal force to loosen any entrapped air and force it to the proximal end of the catheter to ensure it does not migrate into the distal tip near the ultrasound transducer. Other catheter systems may prefill the catheter with fluid during manufacturing and/or prior to shipment. These systems have concerns with shelf life of the fluid as well as shipping time and temperature during which the fluid could freeze and mechanically compromise the fluid seals. These concerns are eliminated by filling the catheter at the point of use with a simple and easy fill method that saves time and results in confidence that the catheter is fluid filled with no entrapped air.

The advantages and features of the invention are further illustrated with reference to the following example, which is not to be construed as in any way limiting the scope of the invention but rather as illustrative of one embodiment of the invention in a specific application thereof.

### Example

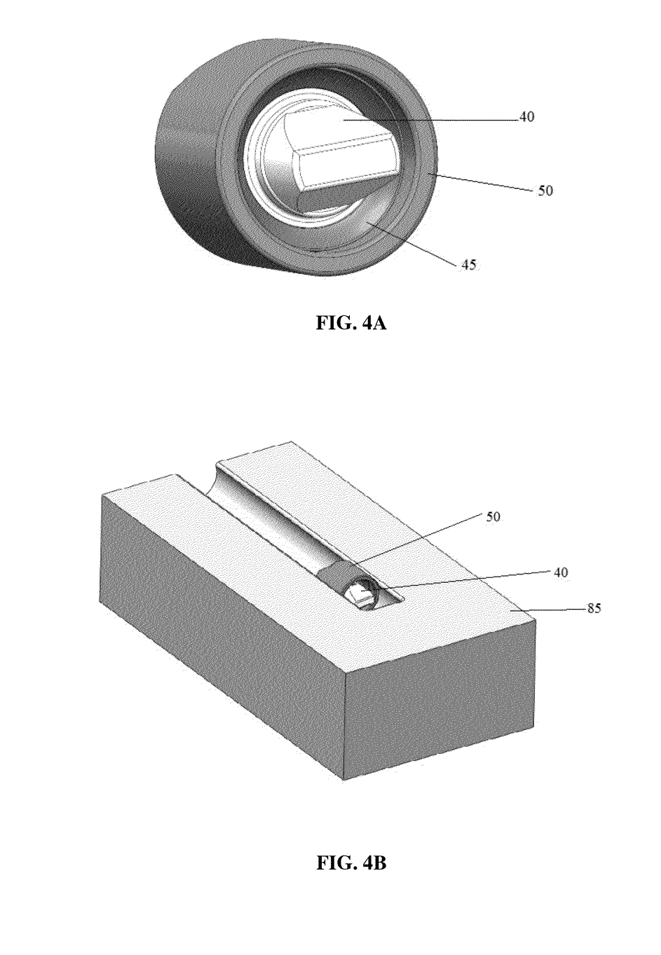

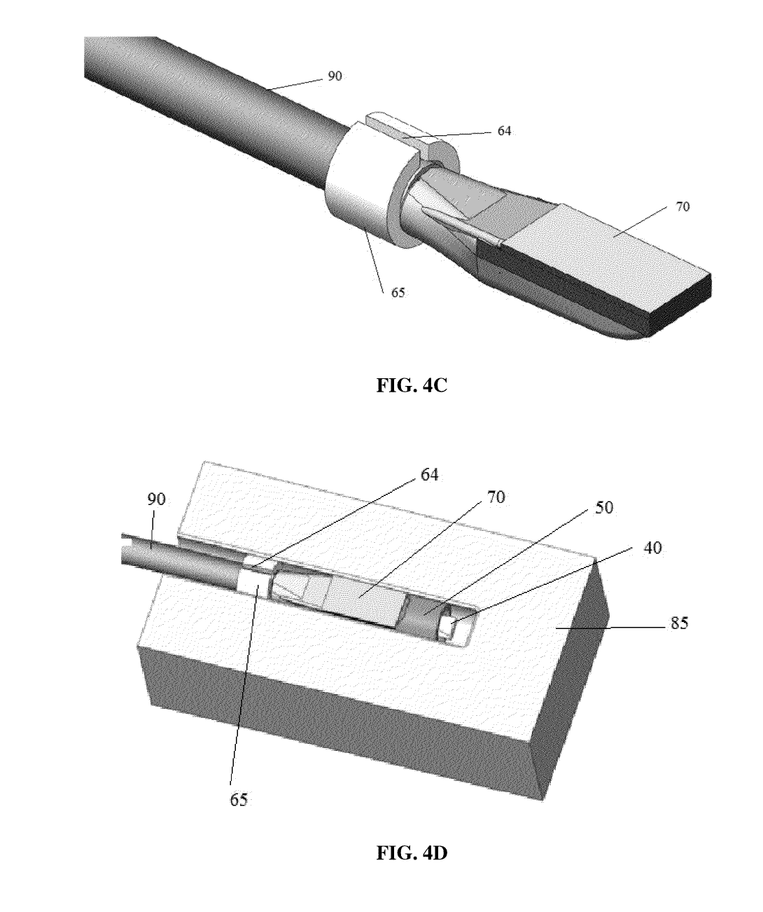

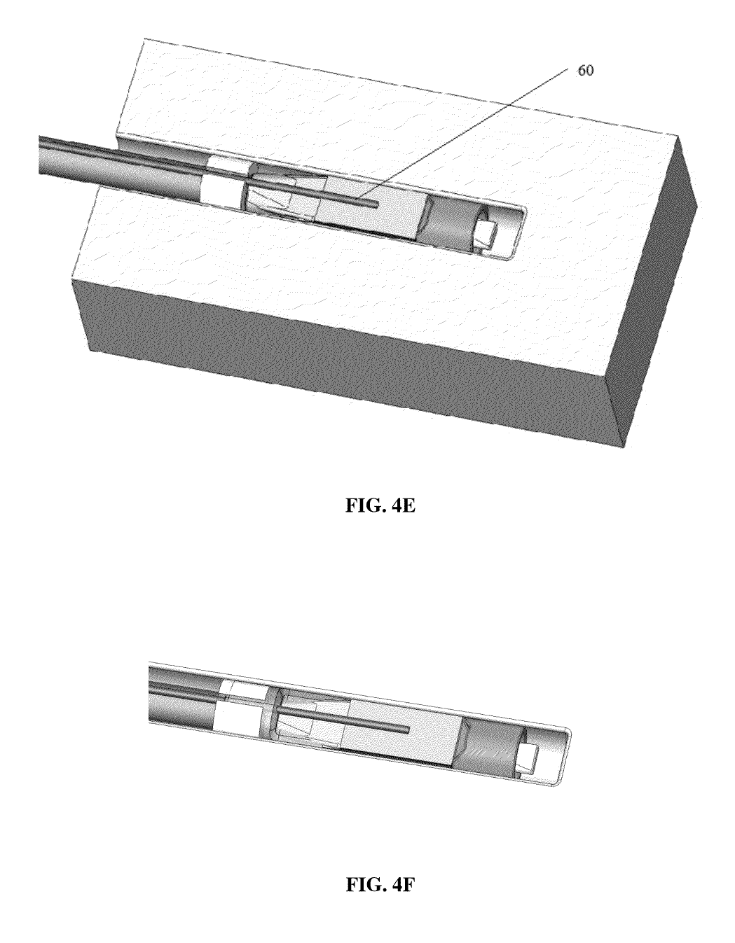

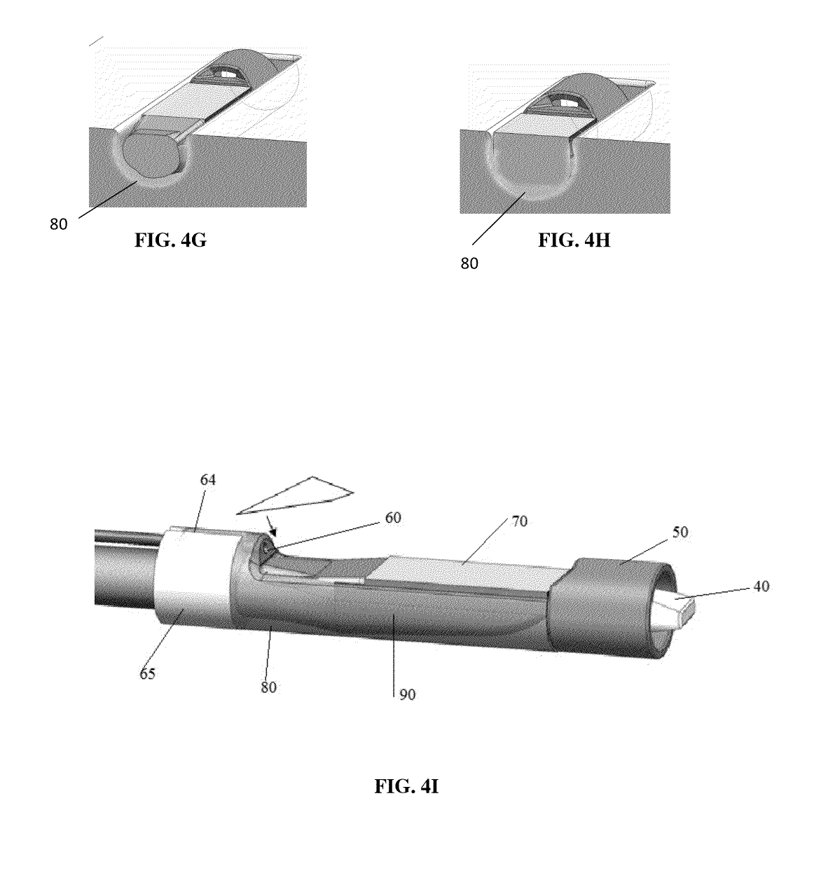

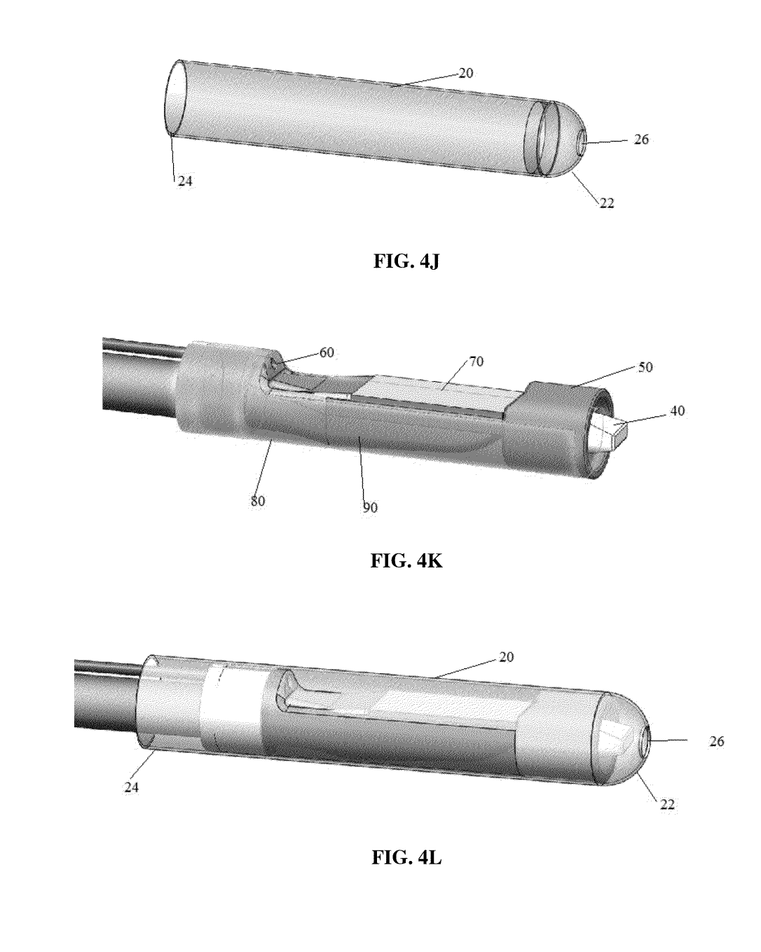

A fluid fillable catheter capsule assembly containing an ultrasound transducer array and electrical wire bundle according to FIG. 1 was constructed according to the following steps, illustrated in FIGS. 4A-4L. A check valve 40 was inserted into the front end of a bulkhead 50, as shown in FIG. 4A such that the check valve 40 was fully engaged within the bulkhead 50. The base of the check valve was placed flat against the seat. The radial orientation was not critical. UV adhesive 45 was applied around the top of the check valve base flange, contacting the inner diameter of the bulkhead seat. The UV adhesive was then cured to create a fluid tight seal between the check valve and the bulkhead and to hold the check valve in place. The UV adhesive used was Loctite 3926 Medical Device Adhesive, part number 36492.

After the UV adhesive was cured, the bulkhead assembly was placed into a potting fixture 85 as shown in FIG. 4B. The through hole in the bulkhead was oriented at the top so that the through hole was in line with the transducer at a later stage of the construction of the fluid fillable catheter capsule.

A C-clip 65 was placed around a wire bundle 90 connected to a transducer 70, as shown in FIG. 4C. The C-clip was pushed up against the transducer, with the gap 64 in the C-clip facing up, perpendicular to the transducer 70 face.

The transducer 70 was placed into the potting fixture 85 close to the proximal end of the distal bulkhead with the transducer face facing upward and flush with the face of the potting fixture 85, as shown in FIG. 4D. Wire bundle 90 extended from the potting fixture 85.

A fill tube 60 was placed on the top of the wire bundle 70, in the gap 64 of the C-clip 65, with its end extending to the surface of the transducer 70, as shown in FIG. 4E.

The space in the potting fixture 85 between the C-clip 65 and the bulkhead 50, and below the transducer 70 was then filled with potting material 80. The potting material 80 was kept off the transducer face. The potting material 80 thus encapsulated the wire bundle 90 below the transducer and filled in around the fill tube 60 and the C-clip 65, as shown in FIG. 4F. Section views of the adhesive filling 80 in the potting fixture are shown in FIGS. 4G and 4H. The adhesive was cured in the potting fixture. The adhesive used had a high enough viscosity to help control the fill and placement in the potting and gluing steps. The potting fixture, the bulkhead and the C-clip were transparent to facilitate the UV curing of the adhesive.

The bulkhead assembly was then removed from the potting fixture and examined to ensure that the components were properly spaced in relation to each other such that the transducer was parallel to the axis of the assembly. The cured adhesive was examined for any gaps in the potted glue surface. The gaps observed were filled with adhesive, the assembly was replaced in the potting fixture and the curing step was repeated. This step was repeated until any gaps in the potted area, bulkhead joint and C-clip/fill tube joint were filled, while care was taken to keep adhesive away from the transducer face and bulkhead lumen.

After the adhesive step was completed, the fill tube was trimmed with a sharp blade to be even with the distal wall and adhesive bead and the excess piece of fill tube was removed, as shown in FIG. 4I.

A capsule 20 sized to encompass the bulkhead assembly as shown in FIG. 4J was placed over the assembly to test the fit. The capsule 20 was transparent and had a small diameter opening 26 in the distal end 22. The proximal end 24 of the capsule was placed over the bulkhead assembly as shown in FIG. 4K. Excess adhesive on the outside diameter of the C-clip of bulkhead was trimmed off where the excess adhesive interfered with the capsule fit. Once the capsule fit was satisfactory, the capsule 20 was glued in place by application of a thin, continuous bead of UV adhesive on the inner diameter of the distal end of the capsule. A small diameter applicator tip was used to apply the adhesive to the distal inner diameter of the capsule, accessing the inner diameter of the capsule through the distal opening of the capsule to prevent getting excess adhesive on the wall of the capsule which the transducer would face. This secured the capsule to the fluid control bulkhead assembly. A thin and even coat of UV adhesive was also applied to the lower surface, below the transducer face of the assembly and completely around the C-clip outer diameter. The resulting catheter capsule assembly is shown in FIG. 4L.

While the disclosure has been set out herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

What is claimed is:

1. A catheter capsule assembly comprising:
a capsule having proximal and distal ends and a lumen therethrough, the capsule sealingly attachable at the proximal end to a catheter body and having an opening in the distal end;
a flow controller sealingly secured to the inner circumference of the lumen of said capsule proximate to the distal end of the capsule;
a sealing element at the proximal end of the capsule,
wherein the flow controller and the sealing element are configured to provide a sealable space therebetween, said sealable space arranged to receive a fluid;
a passageway in the sealing element in open communication with the sealable space; and
a sensor element disposed in the lumen of said capsule in the sealable space.

2. The catheter capsule assembly of claim 1, wherein the flow controller comprises a check valve.

3. The catheter capsule assembly of claim 2, wherein the check valve is sealingly disposed within a bulkhead which is sealingly secured to the inner circumference of the lumen of said capsule.

4. The catheter capsule assembly of claim 1, further comprising a capsule connector at the proximal end of the capsule for sealingly attaching the proximal end of the capsule to a catheter body.

5. The catheter capsule assembly of claim 1, wherein the sensor element comprises a transducer assembly comprising an ultrasound transducer element or array and an electrical connection including at least one of a wire, coaxial wire, wire bundle, cable or flex cable.

6. The catheter capsule assembly of claim 1, wherein the sealing element comprises a c-clip.

7. The catheter capsule assembly of claim 6, wherein the passageway in the sealing element comprises a fill tube.

8. The catheter capsule assembly of claim 1, wherein the flow controller is directly proximate to the distal end of the capsule.

9. A catheter capsule assembly comprising:
a capsule having proximal and distal ends and a lumen therethrough, the capsule sealingly attachable at the proximal end to a catheter body and having an opening in the distal end;
a bulkhead engaging a check valve sealingly secured to an inner circumference of the lumen of said capsule directly proximate to the distal end of the capsule;
a sealing element at the proximal end of the capsule,
wherein the bulkhead engaging a check valve and the sealing element are configured to provide a sealable space therebetween, said sealable space arranged to receive a fluid;
a passageway in the sealing element in open communication with the sealable space; and
an ultrasonic transducer or transducer array disposed in the lumen of said capsule in the sealable space.

10. The catheter capsule assembly of claim 9, wherein the ultrasonic transducer comprises a piezoelectric micromachined ultrasonic transducer.

11. The catheter capsule assembly of claim 9, wherein the ultrasonic transducer comprises a capacitive micromachined ultrasonic transducer.

12. An ultrasound imaging catheter system comprising:
a catheter capsule assembly having a distal end for insertion into and manipulation within a vascularized organism and a proximal end for attaching to a catheter body and providing a user with control over the manipulation of the distal end of the catheter within the vascularized organism;
and a catheter body,
wherein the catheter capsule assembly comprises a capsule having proximal and distal ends and a lumen therethrough, the capsule sealingly attached at the proximal end to the catheter body and having an opening in the distal end; a flow controller sealingly secured to the inner circumference of the lumen of said capsule proximate to the distal end of the capsule; a sealing element at the proximal end of the capsule, wherein the flow controller and the sealing element are configured to provide a sealable space therebetween, said sealable space arranged to receive a fluid; a passageway in the sealing element in open communication with the sealable space; and an ultrasound transducer assembly disposed in the lumen of said capsule in the sealable space.

13. The ultrasound imaging catheter system of claim 12, wherein the flow controller comprises a check valve.

14. The ultrasound imaging catheter system of claim 13, wherein the check valve is sealingly disposed within a bulkhead which is sealingly secured to the inner circumference of the lumen of said capsule.

15. The ultrasound imaging catheter system of claim 12, further comprising a capsule connector at the proximal end of the capsule for sealingly attaching the proximal end of the capsule to the catheter body.

16. The ultrasound imaging catheter system of claim 12, wherein the transducer assembly comprises an ultrasound transducer or array and an electrical connection including at least one of a wire, coaxial wire, wire bundle, cable or flex cable.

17. The ultrasound imaging catheter system of claim 16, wherein the ultrasound transducer is a piezoelectric micromachined ultrasound transducer or a capacitive micromachined ultrasound transducer.

18. The ultrasound imaging catheter system of claim 12, wherein the sealing element comprises a C-clip.

19. The ultrasound imaging catheter system of claim 18, wherein the passageway in the sealing element comprises a fill tube.

20. The ultrasound imaging catheter system of claim 12, wherein the flow controller is directly proximate to the distal end of the capsule.
